(19) United States
(12) Patent Application Publication
(10) Pub. No.: US 2025/0366831 A1
(43) Pub. Date: Dec. 4, 2025

(54) RADIOMICS-BASED ANALYSIS OF INTESTINAL ULTRASOUND IMAGES FOR INFLAMMATORY BOWEL DISEASE

(71) Applicant: CEDARS-SINAI MEDICAL CENTER, Los Angeles, CA (US)

(72) Inventors: Phillip Gu, Los Angeles, CA (US); Jui-Hsuan Chang, Torrance, CA (US); Zhiping Wang, Wayne, PA (US); Xiuzhen Huang, Valencia, CA (US); Dan Carter, Gani Tikva (IL); Dermot McGovern, Los Angeles, CA (US)

(73) Assignee: CEDARS-SINAI MEDICAL CENTER, Los Angeles, CA (US)

(21) Appl. No.: 19/222,133

(22) Filed: May 29, 2025

Related U.S. Application Data
(60) Provisional application No. 63/652,860, filed on May 29, 2024.

(51) Int. Cl. A61B 8/00; A61B 8/08; G06T 7/00; G06V 10/25; G06V 10/26; G06V 10/44; G06V 10/764; G16H 50/30

(52) U.S. Cl. CPC A61B 8/5223; A61B 8/08; A61B 8/461; A61B 8/468; A61B 8/469; G06T 7/0012; G06V 10/25; G06V 10/26; G06V 10/44; G06V 10/764; G16H 50/30; G06T 2207/10132; G06T 2207/30092; G06V 2201/031

(57) ABSTRACT

A system and a method for diagnosis and monitoring of inflammatory bowel diseases (IBD) in a subject are provided. The system includes a memory and a control system. The memory stores machine-readable instructions. The control system includes one or more processors configured to execute the machine-readable instructions. Ultrasound image data associated with the gastrointestinal tract of the subject is received. The received ultrasound image data is processed to output a set of ultrasound image features. The output set of ultrasound image features is received, as an input to an automated algorithm. A set of radiomic features is extracted from the input set of ultrasound image features, using the automated algorithm. The ultrasound image data is classified as normal or abnormal based on the extracted set of radiomic features, the classifying being an output of the automated algorithm.

### CROSS-REFERENCE TO RELATED APPLICATIONS

This application includes a claim of priority under 35 U.S.C. § 119(e) to U.S. provisional patent application No. 63/652,860, filed May 29, 2024, the entirety of which is hereby incorporated by reference.

### TECHNICAL FIELD

This disclosure relates generally to systems and methods for analyzing ultrasound images, and more particularly, to systems and methods for radiomic analysis of intestinal ultrasound images to differentiate between normal and abnormal intestinal ultrasound (IUS) images.

### BACKGROUND

The evolution of ultrasound technology has facilitated the emergence of intestinal ultrasound (IUS) as a valuable, non-invasive, point-of-care tool for monitoring inflammatory bowel diseases (IBD) thereby helping IBD providers make real-time decisions at the bedside. IUS has excellent sensitivity and specificity for detecting inflammation and is a promising research tool for clinical trials and biomarker discovery. Exemplary roles of IUS in diagnostics of bowel diseases are described in Prz Gastroenterol. 2018; 13(1): 1-5. Published online 2018 Mar. 26. doi: 10.5114/pg.2018.74554.

However, the increased adoption of IUS for IBD has uncovered new challenges. First, the growing interest among IBD providers to perform IUS in their practice has led to an increase in novice operators. Considering many IUS parameters for inflammation are, at best, semi-quantitative (except for bowel wall thickness), there is an increased risk of diagnostic errors stemming from IUS image interpretation by inexperienced operators. That is, the IUS technique is highly operator-dependent. This has created a need to support less experienced IUS operators to ensure standardized and accurate image interpretation. Second, IUS is an ideal research tool for imaging biomarker discovery because it is non-invasive and radiation-sparing, but current approaches for biomarker discovery with IUS are confined to parameters defined a priori by human expert consensus. This approach may inadvertently overlook important parameters that are not readily detected by the human eye that could improve biomarker discovery and potentially yield additional insight into biological underpinnings of IBD.

Artificial intelligence (AI) may offer solutions to address the current challenges in IUS. Radiomics, a sub-field of AI, is an objective and quantitative approach to analyze medical imaging through mathematical extraction of spatial distribution of signal intensities and pixel interrelationships. In IBD, investigators have developed radiomic-based models that can detect inflammation and quantify disease severity better than humans. However, these studies are currently limited to computed tomography (CT) and magnetic resonance imaging (MRI), and the role of radiomics for IUS has not been investigated.

IUS for monitoring IBD has uncovered new challenges regarding standardized image interpretation and limitations as a research tool. CT carries significant radiation dose when imaging patients with IBD where subsequent repeat imaging to monitor disease activity is useful, but the cumulative radiation dose from CT is a concern. MRI is relatively expensive and time consuming. Thus, a need exists for monitoring IBD more safely, cost-effectively, and accurately. The present disclosure is directed solving these problems and addressing other needs by applying radiomic analysis of IUS images in IBD, IUS being a safe, fast, inexpensive imaging method with high sensitivity and specificity. Further, IUS is also non-invasive, radiation-free and can be repeated many times. The present disclosure shows that radiomics-based classification model can accurately differentiate between normal and abnormal IUS images.

All publications herein are incorporated by reference to the same extent as if each individual publication or patent application was specifically and individually indicated to be incorporated by reference. In particular, the entire contents of Gu et al., “Radiomics-Based Analysis of Intestinal Ultrasound Images for Inflammatory Bowel Disease: A Feasibility Study,” Crohn's & Colitis 360, vol. 6, issue 2, April 2024, otae034, are hereby incorporated by reference in their entirety for all purposes as if fully set forth herein.

The following description includes information that may be useful in understanding the present invention. It is not an admission that any of the information provided herein is prior art or relevant to the presently claimed invention, or that any publication specifically or implicitly referenced is prior art.

### SUMMARY

In one aspect of the present disclosure, a system for diagnosis and monitoring of inflammatory bowel diseases (IBD) in a subject is provided. In some embodiments, the system includes a memory storing machine-readable instructions; and a control system including one or more processors. The one or more processors are configured to execute the machine-readable instructions to receive ultrasound image data associated with the gastrointestinal tract of the subject; process the received ultrasound image data to output a set of ultrasound image features by segmenting regions of interest (ROIs) or volumes of interest (VOIs) in the received ultrasound image data, wherein the segmenting the ROIS or VOIs in the received ultrasound image data includes segmenting a bowel wall of intestines by using an automated algorithm; receive, as an input to the automated algorithm, the output set of ultrasound image features; extract a set of radiomic features from the input set of ultrasound image features, using the automated algorithm; and classify the ultrasound image data as normal or abnormal based on the extracted set of radiomic features, the classifying being an output of the automated algorithm.

In one aspect of the present disclosure, a method for identifying a subject at high risk for inflammatory bowel diseases (IBD) using radiomics is provided. In some embodiments, the method includes receiving ultrasound image data associated with the gastrointestinal tract of the subject; performing radiomic analysis on the received ultrasound image data by: processing the received ultrasound image data to output a set of ultrasound image features by segmenting regions of interest (ROIs) or volumes of interest (VOIs) in the received ultrasound image data, wherein the segmenting the ROIS or VOIs in the received ultrasound image data includes segmenting a bowel wall of intestines by using an automated algorithm; receiving, as an input to the automated algorithm, the output set of ultrasound image features; extracting a set of radiomic features from the input set of ultrasound image features, using the automated algorithm; and classifying the ultrasound image data as normal or abnormal based on the extracted set of radiomic features, the classifying being an output of the automated algorithm; determining that the subject is at high risk for IBD in response to classifying the ultrasound image data as abnormal; and displaying, on a display device, an indication that the subject is at high risk for IBD.

In one aspect of the present disclosure, a method for distinguishing between normal images and abnormal images using radiomics to monitor inflammatory bowel diseases (IBD) in a subject is provided. In some embodiments, the method performed in a computing system includes receiving ultrasound image data associated with the gastrointestinal tract of the subject; performing radiomic analysis on the received ultrasound image data by: processing the received ultrasound image data to output a set of ultrasound image features by segmenting regions of interest (ROIs) or volumes of interest (VOIs) in the received ultrasound image data, wherein the segmenting the ROIS or VOIs in the received ultrasound image data includes segmenting a bowel wall of intestines by using an automated algorithm; receiving, as an input to the automated algorithm, the output set of ultrasound image features; extracting a set of radiomic features from the input set of ultrasound image features, using the automated algorithm; and classifying the ultrasound image data as normal or abnormal based on the extracted set of radiomic features, the classifying being an output of the automated algorithm; determining that abnormal images are included in the ultrasound image data when a bowel wall thickness is greater than 3 mm and/or when bowel hyperemia with modified Limber score is equal to or higher than 1; determining that all images included in the ultrasound image data are normal when a bowel wall thickness is equal to or less than 3 mm and/or when bowel hyperemia with modified Limber score is less than 1; and displaying, on a display device, an indication that abnormal images are included in the ultrasound image data or all images included in the ultrasound image data are normal. The ultrasound image data includes an intestinal ultrasound (IUS) image including a colon image or an ileum image. The abnormal is defined as average bowel wall thickness >3 mm and/or bowel hyperemia with modified Limber score ≥1.

### BRIEF DESCRIPTION OF THE DRAWINGS

The foregoing and other advantages of the present disclosure will become apparent upon reading the following detailed description and upon reference to the drawings.

FIG. 1 is a functional block diagram of a system for diagnosis and monitoring of inflammatory bowel diseases (IBD) in a subject, according to some implementations of the present disclosure.

FIG. 2 is a flow diagram of a method for diagnosis and monitoring of inflammatory bowel diseases (IBD) in a subject, according to some implementations of the present disclosure.

FIG. 3 is an example mask for an abnormal case.

FIG. 4 is an example cropped image for a convolutional neural network (CNN)-based classification model.

FIG. 5 shows confusion matrix results from testing cohort (n=32).

While the present disclosure is susceptible to various modifications and alternative forms, specific implementations have been shown by way of example in the drawings and will be described in further detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

The present disclosure is described with reference to the attached figures, where like reference numerals are used throughout the figures to designate similar or equivalent elements. The figures are not drawn to scale, and are provided merely to illustrate the instant disclosure. Several aspects of the disclosure are described below with reference to example applications for illustration. It should be understood that numerous specific details, relationships, and methods are set forth to provide a full understanding of the disclosure. One having ordinary skill in the relevant art, however, will readily recognize that the disclosure can be practiced without one or more of the specific details, or with other methods. In other instances, well-known structures or operations are not shown in detail to avoid obscuring the disclosure. The present disclosure is not limited by the illustrated ordering of acts or events, as some acts may occur in different orders and/or concurrently with other acts or events. Furthermore, not all illustrated acts or events are required to implement a methodology in accordance with the present disclosure.

Aspects of the present disclosure can be implemented using one or more suitable processing device, such as general purpose computer systems, microprocessors, digital signal processors, micro-controllers, application specific integrated circuits (ASIC), programmable logic devices (PLD), field programmable logic devices (FPLD), field programmable gate arrays (FPGA), mobile devices such as a mobile telephone or personal digital assistants (PDA), a local server, a remote server, wearable computers, tablet computers, or the like.

Memory storage devices of the one or more processing devices can include a machine-readable medium on which is stored one or more sets of instructions (e.g., software) embodying any one or more of the methodologies or functions described herein. The instructions can further be transmitted or received over a network via a network transmitter receiver. While the machine-readable medium can be a single medium, the term “machine-readable medium” should be taken to include a single medium or multiple media (e.g., a centralized or distributed database, and/or associated caches and servers) that store the one or more sets of instructions. The term “machine-readable medium” can also be taken to include any medium that is capable of storing, encoding, or carrying a set of instructions for execution by the machine and that cause the machine to perform any one or more of the methodologies of the various implementations, or that is capable of storing, encoding, or carrying data structures utilized by or associated with such a set of instructions. The term “machine-readable medium” can accordingly be taken to include, but not be limited to, solid-state memories, optical media, and magnetic media. A variety of different types of memory storage devices, such as a random access memory (RAM) or a read only memory (ROM) in the system or a floppy disk, hard disk, CD ROM, DVD ROM, flash, or other computer readable medium that is read from and/or written to by a magnetic, optical, or other reading and/or writing system that is coupled to the processing device, can be used for the memory or memories.

### Example Systems and Methodologies

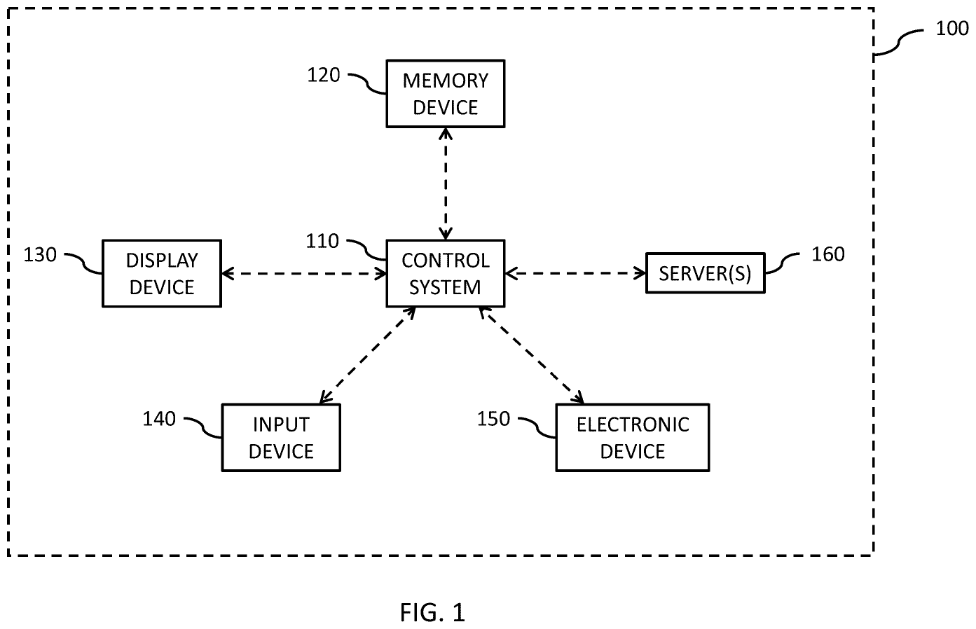

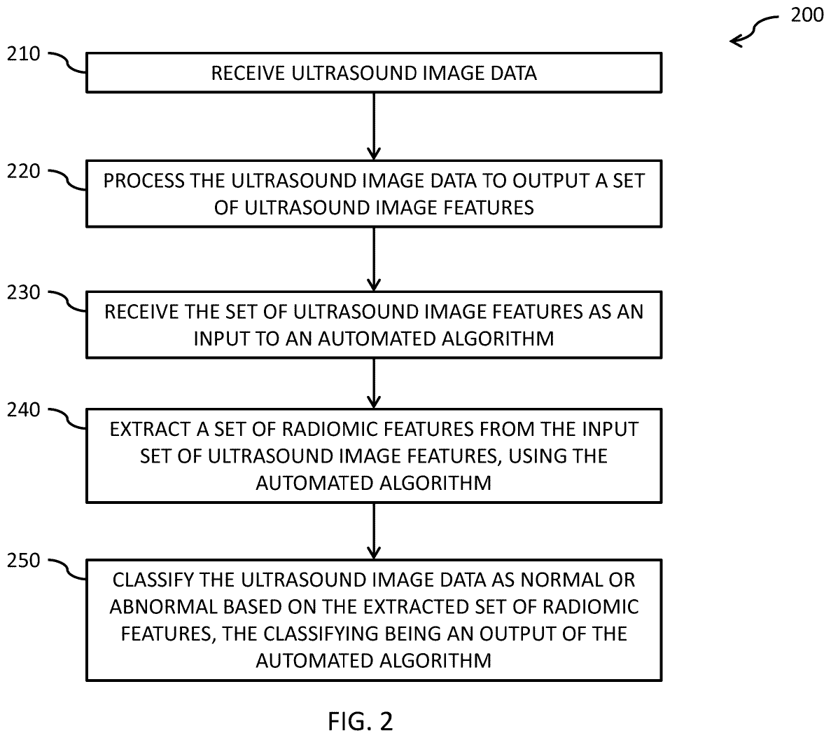

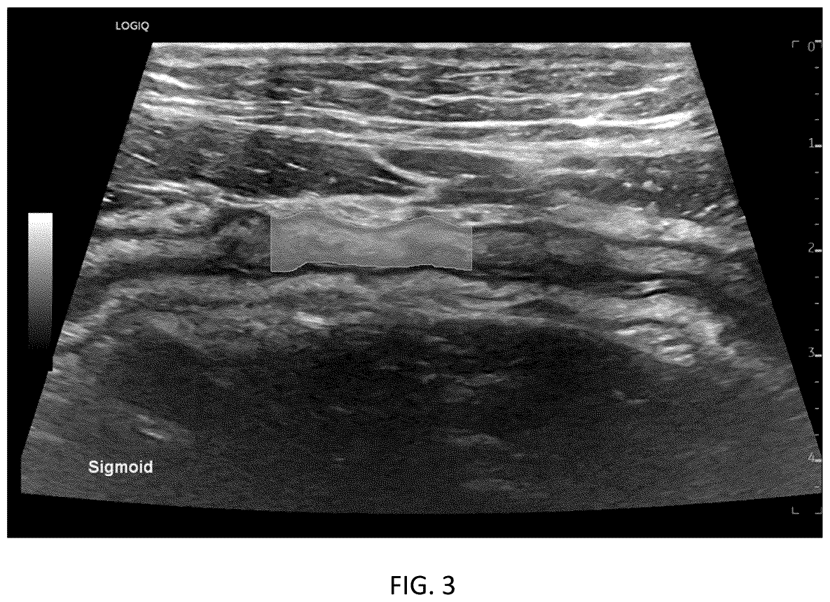

The present disclosure contemplates that a variety of systems can be used to perform various embodiments of the present disclosure. Referring now to FIG. 1, a functional block diagram of a system for diagnosis and monitoring of inflammatory bowel diseases (IBD) in a subject is shown, according to some implementations of the present disclosure. The system 100 can be configured to perform various methods of the present disclosure, including methods 200 and 300 of FIGS. 2 and 3, respectively.

As depicted in FIG. 1, a system 100 includes a control system 110, a memory device 120, a display device 130, and an input device 140. In some implementations, the system 100 also includes an electronic device 150 for generating image data (e.g., an ultrasound machine/transducer). In some implementations, the system 100 further includes one or more servers 160.

The system 100 generally can be used to generate and/or receive a set of device data (e.g., ultrasound image data) associated with a user (e.g., an individual, a person, a patient, etc.) of the electronic device 150. Alternatively or additionally, the system 100 can be used to generate and/or receive a set of clinical data associated with the patient. For example, in some implementations, the set of clinical data can include medical records data (e.g., diagnosis data). The generated and/or received sets of data, in turn, can be analyzed by the system 100 (e.g., using one or more trained algorithms) to predict whether the subject is at high risk for IBD or for diagnosis and monitoring of IBD in the subject.

The control system 110 includes one or more processors. As such, the control system 110 can include any suitable number of processors (e.g., one processor, two processors, five processors, ten processors, etc.). In some implementations, the control system 110 includes one or more processors, one or more memory devices (e.g., the memory device 120, or a different memory device), one or more electronic components (e.g., one or more electronic chips or components, one or more printed circuit boards, one or more power units, one or more graphical processing units, one or more input devices, one or more output devices, one or more secondary storage devices, one or more primary storage devices, etc.), or any combination thereof. In some implementations, the control system 110 includes the memory device 120 or a different memory device, yet in other implementations, the memory device 120 is separate and distinct from the control system 110, but in communication with the control system 110.

The control system 110 generally controls (e.g., actuate) the various components of the system 100 and/or analyzes data obtained and/or generated by the components of the system 100. For example, the control system 110 is arranged to provide control signals to the display device 130, the input device 140, the electronic device 150, or any combination thereof. The control system 110 executes machine readable instructions that are stored in the memory device 120 or a different memory device. The one or more processors of the control system 110 can be general or special purpose processors and/or microprocessors.

While the control system 110 is described and depicted in FIG. 1 as being a separate and distinct component of the system 100, in some implementations, the control system 110 is integrated in and/or directly coupled to the to the display device 130, the input device 140, and/or the electronic device 150. The control system 110 can be coupled to and/or positioned within a housing of to the display device 130, the input device 140, the electronic device 150, or any combination thereof. The control system 110 can be centralized (within one housing) or decentralized (within two or more physically distinct housings).

While the system 100 is shown as including a single memory device 120, it is contemplated that the system 100 can include any suitable number of memory devices (e.g., one memory device, two memory devices, five memory devices, ten memory devices, etc.). The memory device 120 can be any suitable computer readable storage device or media, such as, for example, a random or serial access memory device, a hard drive, a solid state drive, a flash memory device, etc. The memory device 120 can be coupled to and/or positioned within a housing of the to the display device 130, the input device 140, the electronic device 150, the control system 110, or any combination thereof. The memory device 120 can be centralized (within one housing) or decentralized (within two or more physically distinct housings).

The display device 130 of the system 100 is generally used to display text(s) and/or image(s). The image(s) can include still images, video images, projected images, holograms, or the like, or any combination thereof, and/or information regarding to the display device 130, the input device 140, the electronic device 150, or any combination thereof. For example, the display device 130 can provide information regarding the status of the to the display device 130, the input device 140, the electronic device 150 (e.g., the ultrasound machine/transducer), and/or other information. In some implementations, the display device 130 is included in and/or is a portion of the ultrasound machine/transducer. In some implementations, the display device 130 is included in and/or is a portion of the input device 140.

The display device 130 is configured to receive data from the control system 110, and/or the input device 140, and/or the electronic device 150, and/or the server 160. In some implementations, the display device 130 displays input received from the input device 140. In some implementations, data is first sent to the control system 110, which then processes the data and instructs the display device 130 according to the processed data. In some implementations, the display device 130 displays data directly received from the control system 110. In some implementations, the display device 130 displays the texts(s) and/or image(s), and relays the data to the control system 110. In some implementations, the data is then stored in the memory device 120. Examples of such data include a patient profile, ultrasound images, ultrasound image features, a diagnosis prediction, historical medical data, current medical data, or any combination thereof.

The present disclosure also contemplates that more than one display 130 can be used in system 100, as would be readily contemplated by a person skilled in the art. For example, one display can be viewable by a patient, while additional displays are visible to researchers and/or medical professionals and not to the patient. The multiple displays can output identical or different information, according to instructions by the control system 110.

The input device 140 of the system 100 is generally used to receive user input to enable user interaction with the control system 110, the memory device 120, the display device 130, the electronic device 150, or any combination thereof. The input device 140 can include a microphone for speech, a touch-sensitive screen for gesture or graphical input, a keyboard, a mouse, a motion input, or any combination thereof. In some instances, the input device 140 includes multimodal systems that enable a user to provide multiple types of input to communicate with the system 100. The input device 140 can alternatively or additionally include a button, a switch, a dial to allow the user to interact with the system 100. The button, the switch, or the dial may be a physical structure, or a software application accessible via the touch-sensitive screen. In some implementations, the input device 140 may be arranged to allow the user to select a value and/or a menu option. In some implementations, the input device 140 is included in and/or is a portion of the ultrasound machine. In some implementations, the input device 140 is included in and/or is a portion of the display device 130.

In some implementations, the input device 140 includes a processor, a memory, and a display device, that are the same as, or similar to, the processor(s) of the control system 110, the memory device 120, and the display device 130. In some implementations, the processor and the memory of the input device 140 can be used to perform any of the respective functions described herein for the processor and/or the memory device 120. In some implementations, the control system 110 and/or the memory device 120 is integrated in the input device 140.

The display device 130 alternatively or additionally acts as a human-machine interface (HMI) that includes a graphic user interface (GUI) configured to display the image(s) and an input interface. The display device 130 can be an LED display, an OLED display, an LCD display, or the like. The input interface can be, for example, a touchscreen or touch-sensitive substrate, a mouse, a keyboard, or any sensor system configured to sense inputs made by a human user interacting with the system 100 with or without direct user contact/touch.

While the display device 130 and the input device 140 is described and depicted in FIG. 1 as being separate and distinct components of the system 100, in some implementations, the display device 130 and/or the input device 140 are integrated in and/or directly coupled to one or more of the electronic device 150, and/or the control system 110, and/or the memory device 120.

The control system 110 can be communicatively coupled to the memory device 120, the display device 130, the input device 140, and the electronic device 150. Further, the control system 110 can be communicatively coupled to the server 160. For example, the communication can be wired or wireless. The control system 110 is configured to perform any methods as contemplated according to FIG. 2 (discussed further herein). The control system 110 can process and/or store input from the memory device 120, the display 130, the input device 140, and the electronic device 150. In some implementations, the methodologies disclosed herein can be implemented, via the control system 110, on the server 160. It is also contemplated that the server 160 includes a plurality of servers, and can be remote or local. Optionally, the control system 110 and/or the memory device 120 may be incorporated into the server 160.

While the system 100 is shown as including all of the components described herein with respect to FIG. 1, more or fewer components can be included in a system for generating ultrasound image data, analyzing the ultrasound image data using an algorithm, and in turn, predicting whether the subject is at high risk of IBD or diagnosis and monitoring of IBD in the subject. For example, a first alternative system includes the control system 110, the memory device 120, and the electronic device 150. As another example, a second alternative system includes the control system 110, the electronic device 150, and the server 160. As yet another example, a third alternative system includes the control system 110, the memory device 120, the display device 130, and the input device 140. Thus, various systems for identifying individuals at risk for IBD or for diagnosis and monitoring of IBD in individuals can be formed using any portion or portions of the components shown and described herein and/or in combination with one or more other components.

Turning now to FIG. 2, a method 200 for diagnosis and monitoring of IBD is illustrated, according to some implementations of the present disclosure. At step 210, ultrasound image data associated with the gastrointestinal tract of a subject is received, via, for example, a control system 110. Alternatively or additionally, the ultrasound image data associated with the gastrointestinal tract of the subject is generated using an ultrasound transducer.

At step 220, the ultrasound image data is processed, using one or more processors, to output a set of ultrasound image features. In some implementations, the set of ultrasound image features is indicative of a variation in morphology of the intestine (e.g., a size, a shape, a signal intensity, or any combination thereof). In some such implementations, each of the size, shape, and signal intensity is a base class that consists of a plurality of features. For example, there may be hundreds of features that can be extracted on the signal intensity class. Alternatively or additionally, the set of ultrasound image features is indicative of a change in texture of the intestine (e.g., tissue heterogeneity, run length non-uniformity, inverse autocorrelation, long run emphasis, and short run emphasis, or any combination thereof).

At step 230, the set of ultrasound image features is received as an input to an automated algorithm. At step 240, a set of radiomic features is extracted from the input set of ultrasound image features, using the automated algorithm. At step 250, the ultrasound image data is classified as normal or abnormal based on the extracted set of radiomic features. The classifying of the ultrasound image data is an output of the automated algorithm. In some implementations, the set of radiomic features is provided to a machine learning classifier utilized as base models for abnormal classification. In some implementations, the automated algorithm is a machine learning automated algorithm. In some implementations, the machine learning classifier includes Logistic Regression (LR), Decision Tree (DT), Random Forest (RF), Extreme Gradient Boosting (XGB), Multi-Layer Perceptron (MLP), k-Nearest Neighbors (KNN), or any combination thereof.

In some implementations, the ultrasound image data includes a Digital Imaging and Communications in Medicine (DICOM) image and a Neuroimaging Informatics Technology Initiative (NIFTI) segmentation, serving as a region of interest (ROI). The set of radiomic features is extracted from the DICOM image and NIFTI segmentation, using a radiomics features library for the automated algorithm.

In some implementations, the extracted set of radiomic features includes first-order statistics and shape-based metrics providing information about distribution of voxel intensities and an ROI size. In some implementations, the extracted set of radiomic features further includes second-order features encompassing a gray-level co-occurrence matrix (GLCM), gray-level run length matrix (GLRLM), gray-level size zone matrix (GLSZM), neighboring gray-tone difference matrix (NGTDM), and gray-level dependence matrix (GLDM), the second-order features characterizing patterns among pixels and voxels within an ROI.

In some implementations, at least one filter is applied to an original ROI of the ultrasound image data, the at least one filter including one or more of wavelet, square root, gradient magnitude, and a Laplacian of Gaussian. The wavelet decomposes an image into different frequency components, having four sub-bands to represent low/high-frequency information in horizontal/vertical direction. The square root enhances image contrast. The gradient magnitude highlights edges and boundaries. The Laplacian of Gaussian detects regions of rapid intensity change.

In some implementations, the abnormal is defined as average bowel wall thickness of greater than 3 mm and/or bowel hyperemia with modified Limber score of 1 or above 1. In some implementations, the IBD is Crohn's disease or ulcerative colitis.

In some implementations, the ultrasound image data includes an intestinal ultrasound (IUS) image. In some implementations, the IUS image includes a colon image. In some implementations, the IUS image includes an ileum image.

### Example Materials and Methods

Retrospectively analyzing IUS images obtained during routine outpatient visits, inventors developed and tested radiomic-based and CNN-based models to distinguish between normal and abnormal images, with abnormal images defined as bowel wall thickness >3 mm and/or bowel hyperemia with modified Limber score >1 (both are surrogate markers for inflammation). Model performances were measured by area under the receiver operator curve (AUC).

The study was a single-center, retrospective analysis of adult IBD patients (age >18) who underwent IUS during their routine outpatient visit between May 17, 2023 and Nov. 8, 2023. The study was institutional review board (IRB) approved (IRB #3358). Inventors focused the analyses on colon images to avoid confounding from imaging differences with the ileum. Images were included if at least 3 centimeter (cm) colonic bowel wall was visible in the longitudinal axis on the IUS image. Of note, some patients underwent more than one IUS exam during the study period, so images from the same patient but at different time points were also included if the images met the inclusion criteria. For example, IUS images obtain pre- and post-treatment from the same patient could have been included. There were no specific exclusion criteria based on body mass index.

### IUS Protocol and Image Classification

All IUS exams were performed by one IBD specialist who was formally trained by the International Bowel US Group. Subjects were not required to undergo any fasting, bowel preparation or ingestion of oral contrast agents, such as iso-osmolar polyethylene glycol solution (PEG), prior to the exam. The IUS exams were performed using a GE Logiq e10 using a convex transducer (C2-9) for global abdominal assessment and linear transducer (3-12 mHz) for detailed bowel segment measurements and color doppler assessment. Each exam followed a consistent standard technique that included a brief survey of the pelvis followed by a complete grayscale and color doppler evaluation of the colon starting with the sigmoid colon superior to the left iliac vessels in the left lower quadrant of the abdomen until the terminal ileum was identified superior to the right iliac vessels in the right lower quadrant. During these routine exams, standard assessments of the following parameters were obtained and reported for all segments of bowel (sigmoid, descending, transverse, and ascending colon and terminal ileum) based on international expert consensus: 1) bowel wall thickness (BWT, millimeter (mm)) was measured as the average of four measurements, two in the longitudinal plane, and two in the cross-sectional plane from the lumen-mucosa interface to the muscularis propria-serosal interface, 2) bowel wall hyperemia as measured by the presence or absence of color Doppler signal, with a velocity rate of ±5.2 cm/s and graded according the semiquantitative modified Limberg score (scored 0-3). Doppler imaging techniques, such as color Doppler or power Doppler, are tools that provide additional information about vascularisation of the inflamed bowel wall. Based on the intensity of color signals and analysis of Doppler curves with measurement of resistivity index, the examiner can visualize and quantify intestinal wall vascularisation. Presence of inflammatory mesenteric fat, bowel wall echostratification, and presence of reactive mesenteric lymph nodes were also evaluated as part of the exam but were not used for the analysis.

For analyses, colon images were classified as either normal or abnormal, with abnormal defined as average BWT >3 mm and/or modified Limberg score ≥1. These parameters are most important for detecting endoscopically active inflammation.

### Image Post-Processing and Radiomics Feature Extraction

To standardize annotation and reduce bias risk, masks were manually drawn over the bowel wall in the longitudinal axis and were drawn to be 3 cm long with straight edges (FIG. 3). The inner border of the bowel wall was the lumen-mucosa interface, and the outer border of the bowel wall was the submucosa-serosa interface as defined by expert consensus. Radiomic features were extracted from the original Digital Imaging and Communications in Medicine (DICOM) image and Neuroimaging Informatics Technology Initiative (NIFTI) segmentation, serving as the region of interest (ROI) using Pyradiomics library (v 3.0.1). PyRadiomics or Pyradiomics is an open-source python package for the extraction of Radiomics features from 2D and 3D images and binary masks. Radiomics aims to quantify phenotypic characteristics on medical imaging through the use of automated algorithms. Pyradiomics was configured with custom settings, including intensity standardization, outlier removal (for standard deviations>3), and a fixed bin size (binwidth=25) for grey-level discretization to improve the feature repeatability. Additionally, to incorporate further information, four distinct filtering techniques—wavelet, square root, gradient magnitude, and a Laplacian of Gaussian were applied to the original ROI. Wavelet transformation decomposes an image into different frequency components, having four sub-bands to represent low/high-frequency information in horizontal/vertical direction. Other filters, such as the square root filter, enhance image contrast, the gradient magnitude highlights edges and boundaries, and the Laplacian of Gaussian detects regions of rapid intensity change.

A total of 858 radiomic features were extracted, comprising both first-order statistics and shape-based metrics, as well as second-order features. The first-order statistics and shape-based metrics provide insights into the distribution of voxel intensities and ROI size. The second-order features encompass the gray-level co-occurrence matrix (GLCM), gray-level run length matrix (GLRLM), gray-level size zone matrix (GLSZM), neighboring gray-tone difference matrix (NGTDM), and gray-level dependence matrix (GLDM). These second-order features characterize patterns among pixels and voxels within an ROI, considering their spatial arrangement and connectivity. For the subsequent model analysis, only 676 linear independence (Pearson correlation coefficient ≤0.95) features were retained.

### Radiomics Feature Analysis

Given the imbalanced nature of the dataset, inventors utilized balanced bagging with six machine learning classifiers as base models for abnormal classification: Logistic Regression (LR), Decision Tree (DT), Random Forest (RF), Extreme Gradient Boosting (XGB), Multi-Layer Perceptron (MLP), and k-Nearest Neighbors (KNN). Table 1 summarizes each model and their unique strengths and limitations. Because each model has its own learning process and work better for certain data types than others, inventors used these different models to understand which model best fits the radiomics data. The same features were used in all classifier models.

TABLE 1
Summary of definition, strengths, and limitations of each machine-learning classifier models used in the study.

| Model | Definition | Strengths | Limitations |
|---|---|---|---|
| Logistic | Classification model that | Provides probabilities for outcomes. | Assumes a linear relationship between |
| Regression | predicts the probability of a | Coefficients offer insights into feature | features and outcome. |
| | binary outcome based on one or | importance. | Limited flexibility for capturing complex |
| | more predictor variables. | | patterns in data. |
| | | | Sensitive to outliers. |
| | | | Perform may be impacted by highly |
| | | | correlated features. |
| Decision | Non-linear model that divides | Easy to interpret. | Prone to overfitting |
| Tree | data into subsets based on the | Handles both numerical and categorical | Can be sensitive to small data variations. |
| | values of input features, | data. | Small changes in data can result in |
| | forming a tree-like structure | Automatically selects important features. | different trees (instability). |
| | where each internal node | Non-parametric. | Difficulty in capturing relationships |
| | represents a decision based on a | | between features that require interactions. |
| | feature, and each leaf node | | |
| | represents the outcome. | | |
| Random | An ensemble learning method | Reduces overfitting by averaging multiple | More difficult to interpret compared to |
| Forest | that constructs multiple | decision trees. | individual decision trees. |
| | decision trees during training | Good for high-dimensional data. | Potentially poor performance with very |
| | and outputs the mode of the | Robust to outliers and noisy data. | imbalanced datasets. |
| | classes (classification) or the | Provides feature importance estimation. | |
| | average prediction (regression) | | |
| | of the individual trees. | | |
| eXtreme | An ensemble learning method | Highly accurate (frequently wins machine | Requires careful tuning of |
| Gradient | that builds multiple decision | learning competitions). | hyperparameters. |
| Boosting | trees sequentially, each tree | Handles missing data well. | Prone to overfitting if hyperparameters |
| | correcting the errors of the | Regularization techniques reduce | are not tuned properly. |
| | previous one, thereby | overfitting. | Less interpretable compared individual |
| | improving predictive accuracy. | Provides feature importance estimation. | decision trees. |
| | | | May not perform well with noisy or |
| | | | irrelevant features. |
| Multi-Layer | A neural network model | Can learning complex patterns in data. | Requires careful tuning of |
| Perceptron | composed of multiple layers of | Good for large datasets. | hyperparameters. |
| | nodes with each layer fully | Can learn non-linear relationships | Sensitive to feature scaling. |
| | connected to the next one. It | between features and target. | Prone to overfitting, especially with |
| | can learn complex patterns in | Robust to irrelevant features. | insufficient training data. |
| | data through non-linear | | Less interpretable than simpler models |
| | transformations. | | like individual decision trees. |
| k-Nearest | A non-parametric method used | Easy to interpret. | Requires careful selection of the number |
| Neighbors | for classification, where the | No training required; new data can be | of neighbors (k) and distance metric. |
| | output is based on the majority | inputted without retraining. | Sensitive to the scale of features. |
| | vote (classification) or the | Good for small datasets. | Not ideal for high-dimensional data. |
| | average (regression) of the k | | Not good with imbalanced datasets. |
| | nearest data points in the | | |
| | feature space. | | |

Parameter tuning for each model was performed using grid-search, as outlined in Table 2, with an initial focus on optimizing area under the receiver operating characteristic curve (AUC) scores via 5-fold cross-validation. We employed a stratified 5-group fold cross-validation, maintaining an 800%/20% train/test ratio to ensure patient-specific data integrity and minimize bias. Subsequently, the optimal parameters derived from this process were applied to the testing dataset. The performance metric for abnormal classification was evaluated using the area under the receiver operating characteristic curve (AUC). The reported AUC scores were calculated by averaging the results obtained from evaluations across the different shuffle splits. Inventors utilized xgboost libraries (v 2.0.2) for XGB classifiers and the other five classifiers from scikit-learn (v 1.3.0).

TABLE 2
Classifiers and the parameters using in grid-search
(LR = Logistic Regression; DT = Decision
Tree; RF = Random Forest; XGB = Extreme Gradient
Boosting; MLP = Multi-Layer Perceptron; KNN = k-Nearest Neighbours)

| Classifier | Parameter |
|---|---|
| Logistic Regression (LR) | C = [0.1, 1.0, 10.0] penalty = ‘l2’ |
| Decision Tree (DT) | max_depth_values = [1, 2, 3] |
| Random Forest (RF) | n_estimators = [50, 100] |
| | min_samples_split_values = [0.1, 0.5] |
| | max_depth = [1, 2, 3] |
| | sampling_strategy = ‘all’ |
| | replacement = True |
| Extreme Gradient Boosting | min_child_weight_values = [4, 5, 6] |
| (XGB) | max_depth = [1, 2, 3] |
| | learning_rate_values = [0.1, 0.5] |
| Multi-Layer Perceptron (MLP) | hidden_layer_sizes = [(64,), |
| | (64, 32), (64, 64), (64, 128)] |
| | alpha = [100, 0.1, 0.01] |
| | learning_rate_init = [0.001, 0.01] |
| k-Nearest Neighbors (KNN) | n_neighbors = [20, 25, 30] |
| | weights = ‘distance’ |

### Convolutional Neural Network

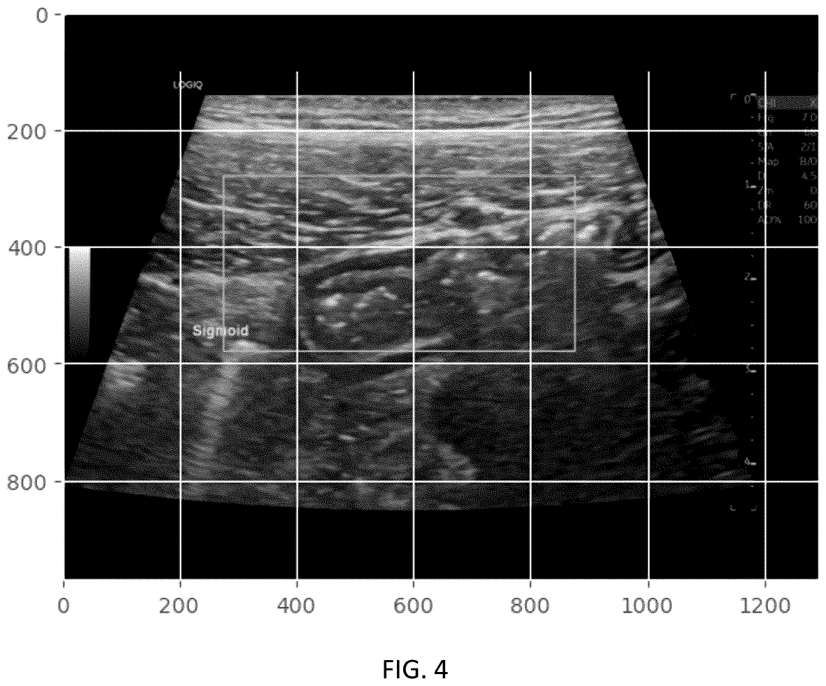

Inventors constructed a custom architecture comprising two base models (EfficientNet-B1 and EfficientNet-B3) as the backbone of CNN. This network was trained using both original images and clinical features, such as age, gender, and race as listed in Table 3. All images were consistently cropped to a size of 600×300 pixels (FIG. 4) based on the input masks. FIG. 4 shows an example of a cropped image for CNN model. To reduce risk of overfitting, inventors applied a series of transformations through the PyTorch transforms module. These transformations include horizontal flips, color jittering augmentation strategies, and normalization. Furthermore, inventors added dropout layers and batch normalization layers for regularization to the CNN model. The inventors' approach involves early stopping, triggered when there is no improvement in the validation set for a specified number of epochs (es_patience), and a scheduled learning rate adjustment (ReduceLROnPlateau). This adjustment dynamically tunes the learning rate during training based on the validation performance.

TABLE 3
Cohort demographics of unique subjects (n = 63)

| | Total unique subjects | |
|---|---|---|
| | (n = 63) | |
| Mean age (SD) | 40.3 | (14.6) |
| Female, n (%) | 34 | (54.0) |
| Race | | |
| White | 49 | (77.8) |
| Black | 4 | (6.3) |
| Asian | 5 | (7.9) |
| Other | 1 | (1.6) |
| IBD Diagnosis, n(%) | | |
| Ulcerative colitis | 28 | (44.4) |
| Crohn's disease | 35 | (55.6) |

SD, standard deviation

During the training phase, inventors calculated the abnormal class weight to adjust the binary cross-entropy with logits loss function, addressing imbalanced image classification. Consequently, inventors employed StratifiedKFold with a parameter (n_splits=5) for evaluation. The primary performance measures were obtained using AUC scores. Throughout our analysis, we utilized the PyTorch library (v1.13.1+cu117) as the CNN framework for model training and evaluation.

### Results

Inventors analyzed 126 images (33% abnormal) obtained from 63 subjects (Table 3).

For this study, 75% of images were used for training and the remaining 25% for testing. The testing and training receiver operating characteristic (ROC) curves and area under the curve (AUC) scores for 6 different machine learning models, including Logistic regression (LR), decision tree (DT), random forest (RF), XGBoost (XGB), K-nearest neighbor (KNN), and multi-layer perceptron (MLP), were obtained. XGB classifier yielded the best performance for classifying normal and abnormal images (average training and test AUC 0.97 and 0.95, respectively, Table 4) followed by RF (average training and test AUC 0.97 and 0.94, respectively).

TABLE 4
Comparative area-under-the curve with 95% confidence interval of
different radiomic-based machine-learning classification models.

| | Linear | | Random | XG | | K-nearest |
|---|---|---|---|---|---|---|
| | regression | Decision | forest | Boost | Neural | neighbor |
| | (LR) | tree (DT) | (RF) | (XGB) | network | (KNN) |
| Train | 0.815 | 0.969 | 0.967 | 0.974 | 0.871 | 0.747 |
| (75% of | (0.673, | (0.951, | (0.946, | (0.955, | (0.8, | (0.7, |
| images) | 0.956) | 0.988) | 0.987) | 0.992) | 0.943 | 0.793) |
| Test | 0.871 | 0.848 | 0.941 | 0.952 | 0.671 | 0.72 |
| (25% of | (0.783, | (0797, | (0.902, | (0.914, | (0.61, | (0.648, |
| images) | 0.958) | 0.899) | 0.979) | 0.990) | 0.732) | 0.792) |

*95% Confidence intervals are shown in parentheses.

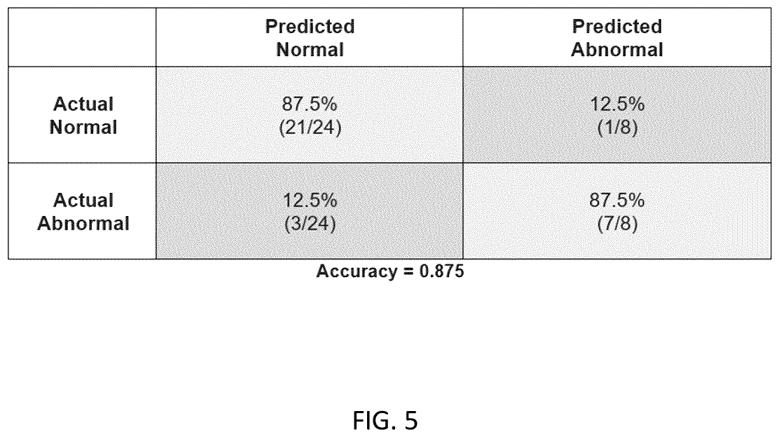

The confusion matrix derived from the testing cohort is presented in FIG. 5. Confusion matrix results from testing cohort (n=32). The model correctly classified 87.5% of the images as normal and 87.5% of images as abnormal. Conversely, 1 normal image was incorrectly classified abnormal while 3 abnormal images were incorrectly classified as normal. The model's sensitivity, specificity, and accuracy were 95.5%, 70.0%, and 87.5%, respectively. The CNN-based classification model yielded an average training and testing AUC of 0.77 and 0.73, respectively.

### DISCUSSION

In this study, inventors demonstrated that radiomic analysis of IUS images is feasible. Inventors also developed radiomic-based classification models that accurately differentiated between normal and abnormal colon IUS images and performed better than a CNN-based model in inventors' cohort.

AI-based applications have the potential to not only improve workflow efficiency but also the accuracy and standardization of imaging interpretation, leading to greater diagnostic precision. Moreover, in oncology, radiomic-based imaging applications have been developed to predict tumor biology. With the growth of IUS for IBD, new challenges have emerged. First, performing IUS is operator-dependent and requires expertise, so the steadily increasing number of inexperienced operators performing IUS for IBD increases the risk of diagnostic error. Second, because it is non-invasive, radiation-sparing, point-of-care tool, IUS offers an ideal research tool for imaging biomarker discovery. However, current approaches are confined to the limited number of IUS parameters used to detect inflammation that have been pre-established by human expert consensus. AI-based approaches can address these challenges with IUS in IBD. Presently, only one study has explored AI in IUS and developed an automated CNN classification model to detect abnormal bowel segments in Crohn's disease. (Carter D, Albshesh A, Shimon C, et al. Automatized Detection of Crohn's Disease in Intestinal Ultrasound Using Convolutional Neural Network. Inflammatory Bowel Diseases 2023; 16:16.) While CNN is excellent for detection and classification with imaging, radiomics allows for a comprehensive analysis of a wide range of features that provide a more detailed and holistic characterization of the underlying biological and pathological processes in the tissue of interest. Radiomics also has the advantage of being able to be integrated with other clinical and “-omic” data for multi-modal analyses to answer a broader spectrum of scientific questions. To inventors' knowledge, this is the first study to conduct radiomic analysis of IUS images. Inventors' findings support the ability of AI to standardize image interpretation and accurately detect inflammation on IUS. Additionally, the inventors' study describes a novel imaging analytical method with IUS for IBD that can expand its research capabilities and can facilitate future imaging biomarkers discovery studies with IUS. These biomarkers can be used to develop innovative multi-modal prediction models and/or yield new insight into the heterogenous nature of IBD when combined with clinical and other “-omic” data.

In conclusion, the study not only demonstrated the feasibility of radiomic analysis of IUS images but also developed a radiomic-based classification model that accurately differentiated normal and abnormal IUS images. With these encouraging results, inventors are working to validate their model in an independent cohort and develop an application for automated bowel wall segmentation to facilitate scalability of their approach in future studies.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present disclosure, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or additionally, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a “data processing apparatus” on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of nonvolatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

### CONCLUSION

One or more elements or aspects or steps, or any portion(s) thereof, from one or more of any of claims below can be combined with one or more elements or aspects or steps, or any portion(s) thereof, from one or more of any of the other claims or combinations thereof, to form one or more additional implementations and/or claims of the present disclosure.

While various examples of the present disclosure have been described above, it should be understood that they have been presented by way of example only, and not limitation. Numerous changes to the disclosed examples can be made in accordance with the disclosure herein without departing from the spirit or scope of the disclosure. Thus, the breadth and scope of the present disclosure should not be limited by any of the above described examples. Rather, the scope of the disclosure should be defined in accordance with the following claims and their equivalents.

Although the disclosure has been illustrated and described with respect to one or more implementations, equivalent alterations and modifications will occur to others skilled in the art upon the reading and understanding of this specification and the annexed drawings. In addition, while a particular feature of the disclosure may have been disclosed with respect to only one of several implementations, such feature may be combined with one or more other features of the other implementations as may be desired and advantageous for any given or particular application.

The terminology used herein is for the purpose of describing particular examples only and is not intended to be limiting of the disclosure. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this disclosure belongs. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

All publications, patents, and patent applications mentioned in this specification are herein incorporated by reference to the same extent as if each individual publication, patent, or patent application was specifically and individually indicated to be incorporated by reference.

What is claimed is:

1. A system for diagnosis and monitoring of inflammatory bowel diseases (IBD) in a subject, the system comprising:
a memory storing machine-readable instructions; and
a control system including one or more processors configured to execute the machine-readable instructions to:
receive ultrasound image data associated with the gastrointestinal tract of the subject;
process the received ultrasound image data to output a set of ultrasound image features by segmenting regions of interest (ROIs) or volumes of interest (VOIs) in the received ultrasound image data, wherein the segmenting the ROIS or VOIs in the received ultrasound image data includes segmenting a bowel wall of intestines by using an automated algorithm;
receive, as an input to the automated algorithm, the output set of ultrasound image features;
extract a set of radiomic features from the input set of ultrasound image features, using the automated algorithm; and
classify the ultrasound image data as normal or abnormal based on the extracted set of radiomic features, the classifying being an output of the automated algorithm.

2. The system of claim 1, further comprising:
an ultrasound transducer configured to generate the ultrasound image data associated with the gastrointestinal tract of the subject; and.
a display device configured to display the generated ultrasound image data.

3. The system of claim 1, wherein the automated algorithm is a machine learning automated algorithm, and wherein the control system including the one or more processors is further configured to execute the machine-readable instructions to determine that the subject whose ultrasound image data is classified as abnormal is at high risk for IBD.

4. The system of claim 3, further comprising a display device, wherein the control system including the one or more processors is further configured to execute the machine-readable instructions to display, on the display device, an indication of whether the subject is at high risk for IBD.

5. The system of claim 1, wherein the control system including the one or more processors is further configured to execute the machine-readable instructions to provide the set of radiomic features to a machine learning classifier utilized as base models for abnormal classification.

6. The system of claim 5, wherein the machine learning classifier includes Logistic Regression (LR), Decision Tree (DT), Random Forest (RF), Extreme Gradient Boosting (XGB), Multi-Layer Perceptron (MLP), k-Nearest Neighbors (KNN), or any combination thereof, or wherein the machine learning classifier includes XGB.

7. The system of claim 1, wherein the automated algorithm is configured with custom settings, including intensity standardization, outlier removal (for standard deviations>3), and a fixed bin size (binwidth=25) for grey-level discretization.

8. The system of claim 1, wherein the abnormal is defined as average bowel wall thickness >3 mm and/or bowel hyperemia with modified Limber score ≥1.

9. The system of claim 1, wherein the ultrasound image data includes an intestinal ultrasound (IUS) image, the IUS image including a colon image or an ileum image; and/or
wherein the IBD is Crohn's disease or ulcerative colitis.

10. A method for identifying a subject at high risk for inflammatory bowel diseases (IBD) using radiomics, the method performed in a computing system comprising:
receiving ultrasound image data associated with the gastrointestinal tract of the subject;
performing radiomic analysis on the received ultrasound image data by:
processing the received ultrasound image data to output a set of ultrasound image features by segmenting regions of interest (ROIs) or volumes of interest (VOIs) in the received ultrasound image data, wherein the segmenting the ROIS or VOIs in the received ultrasound image data includes segmenting a bowel wall of intestines by using an automated algorithm;
receiving, as an input to the automated algorithm, the output set of ultrasound image features;
extracting a set of radiomic features from the input set of ultrasound image features, using the automated algorithm; and
classifying the ultrasound image data as normal or abnormal based on the extracted set of radiomic features, the classifying being an output of the automated algorithm;
determining that the subject is at high risk for IBD in response to classifying the ultrasound image data as abnormal; and
displaying, on a display device, an indication that the subject is at high risk for IBD.

11. The method of claim 10, further comprising providing the set of radiomic features to a machine learning classifier utilized as base models for abnormal classification.

12. The method of claim 11, wherein the machine learning classifier includes Logistic Regression (LR), Decision Tree (DT), Random Forest (RF), Extreme Gradient Boosting (XGB), Multi-Layer Perceptron (MLP), k-Nearest Neighbors (KNN), or any combination thereof or wherein the machine learning classifier includes XGB.

13. The method of claim 10, further comprising drawing masks on the ultrasound image data over a bowel wall in a longitudinal axis, the masks drawn to be 3 centimeter (cm) long with straight edges.

14. The method of claim 13, wherein an inner border of the bowel wall is the lumen-mucosa interface, and an outer border of the bowel wall is the submucosa-serosa interface.

15. The method of claim 10, wherein the automated algorithm is configured with custom settings, including intensity standardization, outlier removal (for standard deviations>3), and a fixed bin size (binwidth=25) for grey-level discretization.

16. The method of claim 10, wherein the abnormal is defined as average bowel wall thickness >3 mm and/or bowel hyperemia with modified Limber score >1.

17. The method of claim 10, wherein the ultrasound image data includes an intestinal ultrasound (IUS) image including a colon image or an ileum image, and/or the IBD is Crohn's disease or ulcerative colitis.

18. A method for distinguishing between normal images and abnormal images using radiomics to monitor inflammatory bowel diseases (IBD) in a subject, the method performed in a computing system comprising:
receiving ultrasound image data associated with the gastrointestinal tract of the subject;
performing radiomic analysis on the received ultrasound image data by:
processing the received ultrasound image data to output a set of ultrasound image features by segmenting regions of interest (ROIs) or volumes of interest (VOIs) in the received ultrasound image data, wherein the segmenting the ROIS or VOIs in the received ultrasound image data includes segmenting a bowel wall of intestines by using an automated algorithm;
receiving, as an input to the automated algorithm, the output set of ultrasound image features;
extracting a set of radiomic features from the input set of ultrasound image features, using the automated algorithm; and
classifying the ultrasound image data as normal or abnormal based on the extracted set of radiomic features, the classifying being an output of the automated algorithm;
determining that abnormal images are included in the ultrasound image data when a bowel wall thickness is greater than 3 mm and/or when bowel hyperemia with modified Limber score is equal to or higher than 1;
determining that all images included in the ultrasound image data are normal when a bowel wall thickness is equal to or less than 3 mm and/or when bowel hyperemia with modified Limber score is less than 1; and
displaying, on a display device, an indication that abnormal images are included in the ultrasound image data or all images included in the ultrasound image data are normal, wherein the ultrasound image data includes an intestinal ultrasound (IUS) image including a colon image or an ileum image, and
wherein the abnormal is defined as average bowel wall thickness >3 mm and/or bowel hyperemia with modified Limber score ≥1.

19. The method of claim 18, wherein the automated algorithm is a machine learning automated algorithm, the method further comprising providing the set of radiomic features to a machine learning classifier utilized as base models for abnormal classification, and wherein the machine learning classifier includes Logistic Regression (LR), Decision Tree (DT), Random Forest (RF), Extreme Gradient Boosting (XGB), Multi-Layer Perceptron (MLP), k-Nearest Neighbors (KNN), or any combination thereof, or wherein the machine learning classifier includes XGB.

20. The method of claim 18, further comprising drawing masks on the ultrasound image data over a bowel wall in a longitudinal axis, the masks drawn to be 3 centimeter (cm) long with straight edges,
wherein an inner border of the bowel wall is the lumen-mucosa interface, and an outer border of the bowel wall is the submucosa-serosa interface,
wherein the ultrasound image data includes a Digital Imaging and Communications in Medicine (DICOM) image and a Neuroimaging Informatics Technology Initiative (NIFTI) segmentation, serving as a region of interest (ROI), and
wherein the method further comprises extracting the set of radiomic features from the DICOM image and NIFTI segmentation, using a radiomics features library for the automated algorithm.
